(19) United States
(12) Patent Application Publication
(10) Pub. No.: US 2021/0335502 A1
(43) Pub. Date: Oct. 28, 2021

(54) MULTI-MODALITY ELECTRONIC INVITE ROUTING SYSTEM AND PROCESS FOR TELEHEALTH LANGUAGE INTERPRETATION SESSION

(71) Applicant: Language Line Services, Inc., Monterey, CA (US)

(72) Inventors: Jeffrey Cordell, Carmel, CA (US); Lindsay D'Penha, Carmel, CA (US); Adam Caldwell, Carmel Valley, CA (US); James Boutcher, Carmel, CA (US); Jordy Boom, Marina, CA (US)

(73) Assignee: Language Line Services, Inc., Monterey, CA (US)

(21) Appl. No.: 16/858,407

(22) Filed: Apr. 24, 2020

(51) Int. Cl. G16H 80/00; G16H 40/67; G16H 40/20; G06F 40/58

(52) U.S. Cl. CPC G16H 80/00; G06F 40/58; G16H 40/20; G16H 40/67

(57) ABSTRACT

A computer-implemented process receives, during a telehealth conference, an interpreter invitation according to a modality generated through a user interface at a provider computing device operated by the healthcare provider. An example of a modality is a web form, email, hyperlink, phone call via an interactive voice recognition system, mobile software application, desktop tool tray software application, text message, calendar invitation, QR code, or the like. The computer-implemented process routes the interpreter invitation from the telehealth platform to a language interpretation platform that extracts an invitation request payload from the interpreter invitation and routes the invitation request payload, through an automatic call distribution network, to a next available language interpreter operating a language interpreter workstation such that the language interpreter workstation automatically selects a plugin according to a teleconference platform indicated by the invitation request payload.

### BACKGROUND

### 1. Field

This disclosure generally relates to the field of language interpretation. More particularly, the disclosure relates to computer-implemented language interpretation systems for telehealth.

### 2. General Background

With adverse consequences of viral outbreaks, such as the coronavirus known as “COVID-19,” various healthcare providers (e.g., hospitals, doctor's offices, etc.) have attempted to comply with social distancing mandates and guidelines through various telehealth configurations. In other words, telehealth configurations are used to remotely provide healthcare services to patients, not physically situated in front of the healthcare provider (e.g., doctor, pharmacist, nurse, lab technician, physical therapist, acupuncturist, chiropractor, etc.). For example, a patient may be conveniently located in his or her own home, and have a telehealth video conference with his or her doctor to describe symptoms of a new, or ongoing, medical situation. Alternatively, the patient may be situated in a different room, at the physical location of the healthcare provider, than that of the healthcare provider. As another example, the telehealth configuration may be utilized by healthcare providers to confer with one another regarding various matters (e.g., an ongoing case, research, etc.). (The term “telehealth” is intended to include a wide variety of healthcare services, such as medical consultations, examinations, appointments, reminders, education, monitoring, and the like, as well as telemedicine services including clinical services such as diagnostics.)

Telehealth configurations are also utilized for other purposes besides social distancing. For example, a patient may live in a particular geographic area, which has limited access to the medical services for which the patient is in need. Accordingly, a telehealth configuration may help that patient obtain access to medical services that may be difficult for that patient to obtain otherwise.

With the increased necessity, for the foregoing reasons, of telehealth configurations, healthcare providers have been attempting to adopt telehealth technologies as rapidly as possible. Yet, conventional telehealth systems are typically configured only for the same human-spoken language (i.e., a language that is traditionally spoken by a group of people originating from a particular geographical location, country, or region) telehealth communication. For example, a patient speaking English may schedule a telehealth video conference with a physician, who also speaks English. However, if a Spanish-speaking patient, also called a limited English proficiency speaker (“LEP”), wants to schedule a telehealth video conference with his or her physician, current telehealth systems are not adequately equipped to provide such a communication.

As a result, conventional telehealth configurations have limited efficacy, particularly with respect to LEP patients participating in telehealth sessions with English-speaking healthcare providers, thereby leaving many LEPs without the benefit of telehealth medical services.

### SUMMARY

A computer-implemented process receives, during a telehealth conference between a healthcare provider speaking a first language and a patient speaking a second language, an interpreter invitation according to a modality generated through a user interface at a provider computing device operated by the healthcare provider.

An example of a modality is a web form, email, hyperlink, phone call via an interactive voice recognition system (“IVR”), mobile software application, desktop tool tray software application, text message, calendar invitation, QR code, or the like.

Furthermore, the computer-implemented process routes the interpreter invitation from the telehealth platform to a language interpretation platform that extracts an invitation request payload from the interpreter invitation and routes the invitation request payload, through an automatic call distribution (“ACD”) network, to a next available language interpreter operating a language interpreter workstation such that the language interpreter workstation automatically selects a plugin according to a teleconference platform indicated by the invitation request payload.

Finally, the computer-implemented process establishes an updated telehealth conference that modifies the telehealth conference to include the next available language interpreter to perform real-time language interpretation for a communication between the healthcare provider and the patient.

Alternatively, a computer program product may have a computer readable storage device with a computer readable program stored thereon that implements the functionality of the aforementioned process. As yet another alternative, a system may implement the process via various componentry, such as a receiver, a routing engine, and a processor.

### BRIEF DESCRIPTION OF THE DRAWINGS

The above-mentioned features of the present disclosure will become more apparent with reference to the following description taken in conjunction with the accompanying drawings wherein like reference numerals denote like elements and in which:

FIG. 1 illustrates a multi-modality electronic invite routing system that may be utilized to initiate a telehealth language interpretation session.

FIG. 2A illustrates an example of a telehealth graphical user interface (“GUI”) being overlain over a messaging software application, such as an email client, on a display screen of the healthcare provider computing device.

FIG. 2B illustrates an example of the messaging software application allowing the healthcare provider to send a message, such as an email to the patient to schedule a teleconference.

FIG. 2C illustrates an example of a telehealth meeting GUI.

FIG. 2D illustrates a language interpreter invite widget that may be rendered in addition to the teleconference telehealth meeting GUI.

FIG. 2E illustrates the telehealth meeting GUI depicting an updated telehealth meeting.

FIG. 3A illustrates an interpreter GUI that may be rendered on the interpreter computing device to allow the language interpreter to perform language interpretation via a telehealth session.

FIG. 3B illustrates the language interpreter, if available, receiving an interpreter invite, as indicated by the interpreter invite notification in the GUI illustrated in FIG. 3B.

FIG. 3C illustrates an interpreter teleconference window that is automatically rendered upon acceptance of the interpreter invite illustrated in the interpreter invite notification of FIG. 3B.

FIG. 3D illustrates the interpreter window, the healthcare provider window, and the patient window that are rendered during the updated telehealth conference after the joining of the language interpreter.

FIG. 4 illustrates an extraction configuration in which the language interpretation platform performs extraction of an interpreter request payload from the interpreter invite that is sent from the healthcare provider computing system.

FIG. 5 illustrates a plugin selection configuration that allows the interpreter workstation to automatically select, utilizing a plugin selection engine, a plugin corresponding to the telehealth platform that is configured for use with the healthcare provider computing system.

FIG. 6A illustrates a system configuration for the healthcare provider computing device.

FIG. 6B illustrates a system configuration for the telehealth platform.

FIG. 6C illustrates a system configuration for the language interpretation platform.

FIG. 6D illustrates a system configuration for the language interpreter workstation.

FIG. 7 illustrates a process that may be utilized to provide multi-modality electronic invite routing.

### DETAILED DESCRIPTION

A multi-modality electronic invite routing system and process are provided to initiate a telehealth language interpretation session between a healthcare provider, patient, and language interpreter. In particular, a telehealth computer-implemented system, which may be utilized by a healthcare provider, may send an electronic invite, via one of a plurality of modalities (e.g., web form, email, hyperlink, phone call via IVR, mobile software application, desktop tool tray software application, text message, calendar invitation, QR code, or the like) to a language interpretation computer-implemented platform (e.g., a computerized contact center). Subsequently, the language interpretation computer-implemented platform may then route the electronic invite to the next available language interpreter. In essence, the multi-modality electronic invite routing system automatically allows a language interpreter to join a live telehealth communication (e.g., video conference, telephone call, etc.) without additional software tools or steps. For instance, a language interpreter workstation may automatically select which telehealth system plugin software should be loaded for a given telehealth session.

The invite may be parsed by the language interpretation computer-implemented platform to extract the data most pertinent to the language interpreter, and then may be presented to the language interpreter via a universal GUI at the language interpreter workstation. In other words, no matter the modality of the original invite (web form, text message, etc.), the language interpreter views the electronic invite via the same GUI. As an alternative, the language interpretation computer-implemented platform may send the electronic invite in its original form to the language interpreter. For instance, the language interpretation computer-implemented platform may route a text message from a mobile computing device of the healthcare provider to a mobile computing device of the language interpreter.

In one embodiment, the multi-modality electronic invite routing system is a real-time, on-demand language interpretation system that allows for a language interpreter to be joined to a telehealth session in real-time (measured as a humanly imperceptible delay), or in substantially real-time (measured as a slight humanly perceptible delay (e.g., one to ten seconds), from the time that the electronic invite is sent to the time that the language interpreter joins the telehealth virtual meeting. In another embodiment, the multi-modality electronic invite routing system is a reservation-based language interpretation system that allows sending of an electronic reservation request for a language interpretation session with a language interpreter.

An example of the language interpretation to be performed by a language interpreter is from a first human-spoken language (e.g., English) to a second human-spoken language (e.g., Spanish); and vice-versa. Another example of the language interpretation may be from a human-spoken language to a sign language.

Moreover, for illustrative purposes, the language interpreter provided for herein is described and illustrated as a human language interpreter that performs the language interpretation via a computer implemented workstation (e.g., desktop computer, laptop, tablet device, smartphone, etc.). Alternatively, the language interpreter may be a machine interpreter that is controlled by an artificial intelligence (“AI”) system, which may manipulate a virtual avatar, via video and audio outputs, during the telehealth virtual meeting.

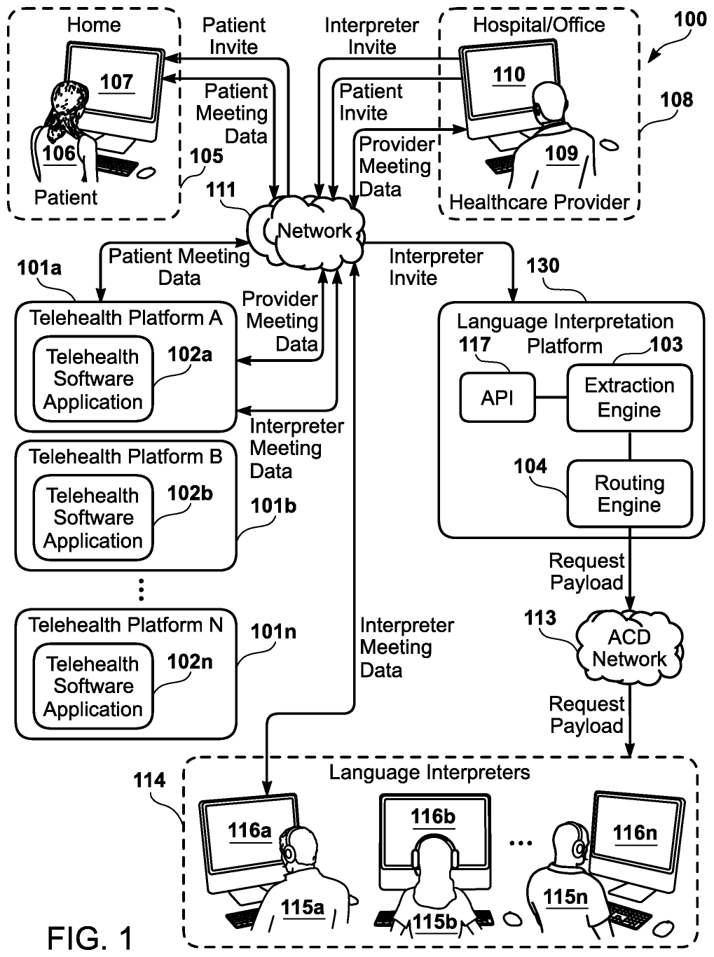

FIG. 1 illustrates a multi-modality electronic invite routing system 100 that may be utilized to initiate a telehealth language interpretation session. In essence, the multi-modality electronic invite routing system 100 is an open framework that allows any invite-based video conferencing system to distribute an electronic invite request to a group of language interpreters so that one of them may be selected to perform language interpretation between a healthcare provider 109 and a patient 106, such as an LEP.

Initially, the healthcare provider 109 may schedule a telehealth session with the patient 106; or vice versa. For example, a healthcare provider 109, or a staff member in a hospital/office 108 of the healthcare provider 109, may utilize a healthcare provider computing device 110 to send a patient invite via a network 111 to a patient computing device 107 of a patient 106. The patient invite may be sent in electronic form (e.g., email, calendar invite, etc.), and the patient 106 may send an acceptance to the healthcare provider 109. The patient invite may have login information (e.g., hyperlink, meeting credentials, etc.) for the patient 106 to use to login to a teleconference, which may be video-based, at a designated day and time that may be indicated by the patient invite.

At the time of the scheduled teleconference, the patient 106 may activate the teleconference by utilizing the data provided in the patient invite. For example, the patient 106 may click a hyperlink within the patient invite that has information specific to a particular telehealth platform 101a, as well as the particular teleconference in which the patient 106 is scheduled to participate with the healthcare provider 109. In one embodiment, the healthcare provider computing device 110 may be configured to establish a teleconference with a specific telehealth platform 101a, which may be selected from a plurality of telehealth platforms 101a-n. For example, each of the telehealth platforms 101a-n may provide video-based teleconferencing via a web portal; however, the particular telehealth platform 101a utilized by the specific healthcare provider 109 may vary from other health care providers, or may even vary by the same healthcare provider 109 based upon different features and services provided by the different telehealth platforms 101a-n.

In particular, the healthcare provider computing device 110, or system, may be configured to utilize a telehealth software application 102a hosted by the telehealth platform 101a to invoke a teleconferencing session. The telehealth software application 102a may be utilized by the healthcare computing device 110 and the patient computing device 107 to establish and join the telehealth conference. Upon establishing the teleconference, each of the healthcare computing device 110 and the patient computing device 107 may generate a user interface, such as a GUI, that allows each of the healthcare provider 109 and the patient 106 to view and hear each other during the teleconference. Various meeting data may be sent from the telehealth platform 101a to the healthcare computing device 110 and the patient computing device 107 to allow for visual and/or audio output at the healthcare computing device 110 and the patient computing device 107.

After the initiation of the teleconference, the healthcare provider 109 and/or the patient 106 may determine that a language interpreter is needed to effectively communicate during the teleconference. Upon such a determination, the healthcare provider computing device 110 may generate an interpreter invite through a language interpretation platform 130, such as through an application programming interface (“API”) 117 of the language interpretation platform 130. For example, the healthcare provider computing device 110 may display a first GUI (e.g., first web portal) corresponding to the telehealth platform 101a and a second GUI (e.g., second web portal, widget, etc.) in which the healthcare provider 109 may request a language interpreter to join the live, ongoing teleconference displayed by the first GUI. Alternatively, the telehealth platform 101a may have an integrated plugin or feature that allows the healthcare provider 109 to generate an interpreter invite directly from the GUI corresponding to the telehealth software application of the telehealth platform 101a.

Irrespective of whether or not the interpreter invite generation widget is integrated within the telehealth platform 101a, the interpreter invite generation widget allows the healthcare provider 109 to generate an interpreter invite from one of a number of different modalities, such as web form, email, hyperlink, phone call via IVR, mobile software application, desktop tool tray software application, text message, calendar invitation, QR code, or the like. In essence, the language interpretation platform 130 allows the healthcare provider 109 to select the modality that is most convenient for him or her to request a language interpreter during the teleconference. For example, one particular healthcare provider may find that having the interpreter invite generation widget in another GUI window beside the GUI window for the telehealth software application 102a is most convenient; whereas another health care provider may find that sending a text message from his or her mobile computing device while participating in the teleconference at the healthcare provider computing device 110 (e.g., desktop computer), is most convenient to request the language interpreter during the teleconference.

Subsequent to receiving the interpreter invite from the healthcare provider computing device 110, the language interpretation platform 130 may utilize an extraction engine 103 to extract an invitation request payload from the interpreter invitation. For example, the extraction engine 103 may extract a customer identifier, a language request, and a telehealth meeting identifier from the interpreter invitation. In essence, the extraction engine 103 normalizes data, received from multiple possible modalities, into a universal format. For example, some telehealth platforms are only capable of sending email requests with fixed information in the body of the email. The extraction engine 103 parses the email, and determines the account and languages of the healthcare provider 109 and/or the patient 106 based on the send and to email addresses, respectively. Furthermore, the extraction engine 103 parses the email for the telehealth meeting identifier. Given that email formats can rapidly change, the extraction engine 103 may be encapsulated in an adapter or wrapper, which constitutes intermediary software code that can perform the extraction of the extraction engine 103 for varying email formats.

Based on the information identified (e.g., language for language interpretation) in the invitation request payload, the language interpretation platform 130 then routes the invitation request payload to the next available language interpreter 115a operating a language interpreter workstation 116a, from a queue 114 of available language interpreters 115a-115n operating language interpreter workstations 116a-n, who is qualified to perform the language interpretation in the language identified by the invitation request payload. As an example, the language interpretation platform 130 may automatically route the normalized invitation request payload via an ACD network 113. By having the invitation request payload normalized, the various language interpretation workstation 116a-n may have a universal GUI that may display the data from the invitation request payload in the same format, while still allowing for the interpreter invitation to be initially sent by the healthcare provider 109 and/or the patient 106 via various different modalities.

Additionally, the invitation request payload may include data that allows the language interpreter workstation 116a to automatically join the teleconference on the particular telehealth platform 101a utilized by the healthcare provider 109 and the patient 106. For instance, the language interpreter workstation 116a may have a plurality of preinstalled plugins, each corresponding to a particular telehealth platform. Based on the telehealth platform identified in the invitation request payload, the language interpreter workstation 116a is able to automatically select and load the corresponding plugin for to join the live, ongoing telehealth virtual meeting. In essence, the automatic selection of the plugin allows for a real-time, or substantially real-time, joining of the teleconference by the language interpreter 115a, thereby improving the processing time for invocation of a telehealth meeting. Accordingly, the language interpreter workstation 116a may have a software application, which may be local or browser-based, that automatically deciphers the invitation request payload, automatically recognizes the telehealth platform based on the automatic deciphering, and automatically launches a corresponding software application that automatically joins the next available language interpreter to the updated telehealth conference. Alternatively, the language interpreter may manually join the teleconference based upon information from the normalized invitation payload request viewable by the language interpretation workstation.

Furthermore, the language interpretation platform 130 may monitor a start time and an end time of the updated telehealth conference (i.e., from the time that the language interpreter joins the telehealth conference to the time that he or she exits the telehealth conference); such monitoring allows for billing for the language interpretation and/or updating availability of the language interpreter. In addition, the language interpretation platform 130 may monitor the updated telehealth conference, via an audio-only system, to maintain quality control of the language interpretation.

Although the foregoing configuration is described with respect to the healthcare provider 109 scheduling the teleconference with the patient 106 and sending the interpreter invitation, in the alternative, the patient 106 may perform one or both tasks.

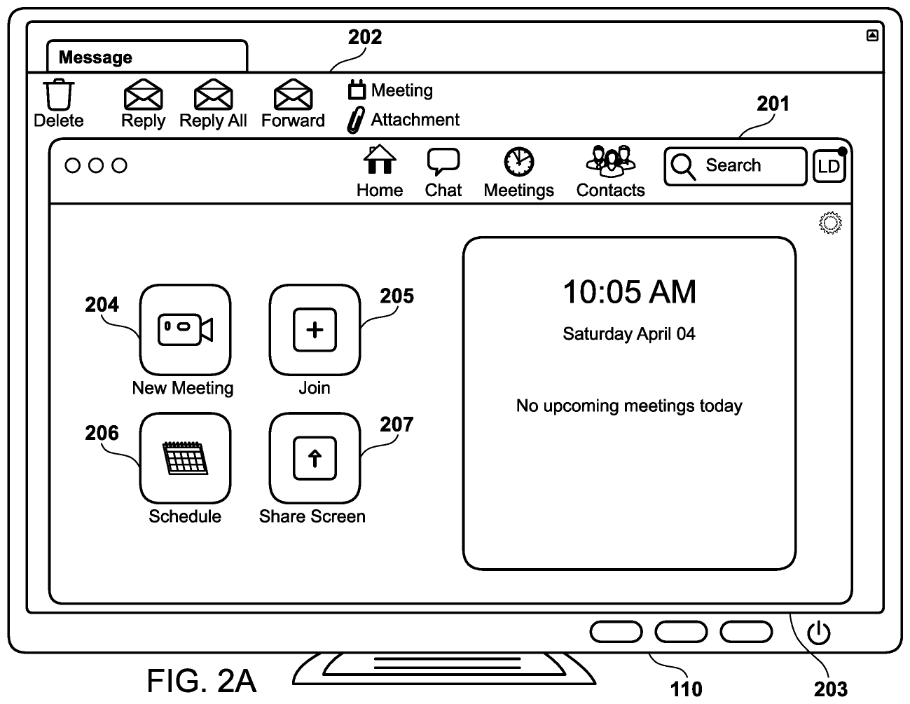

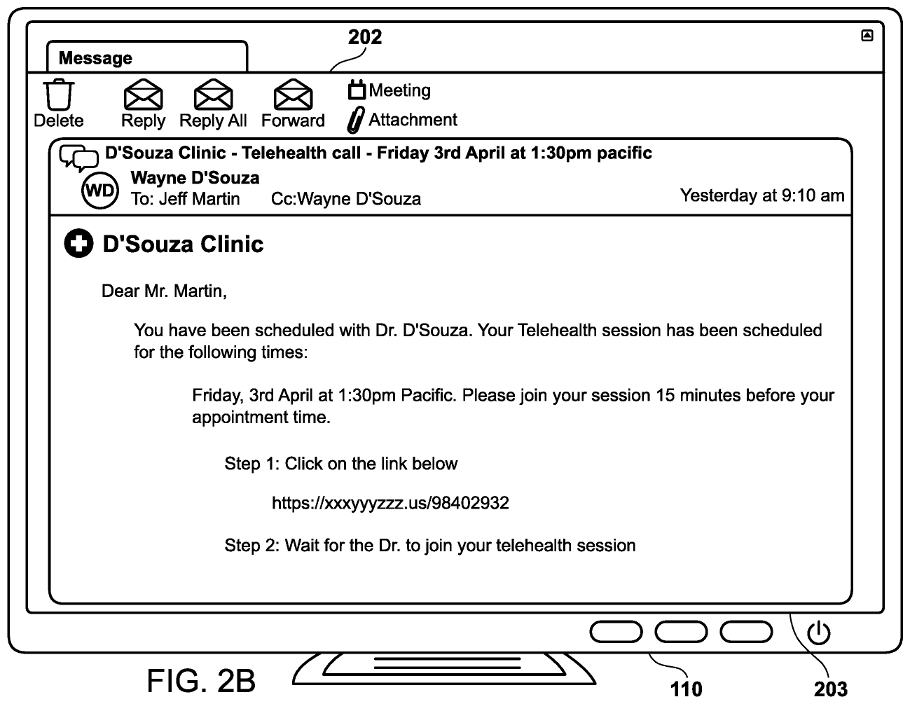

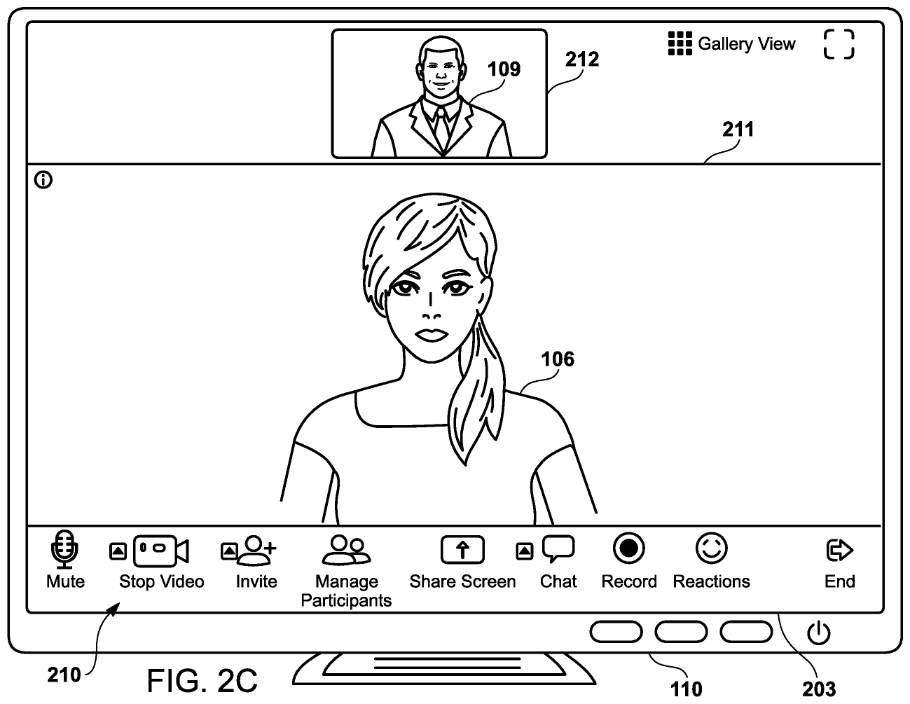

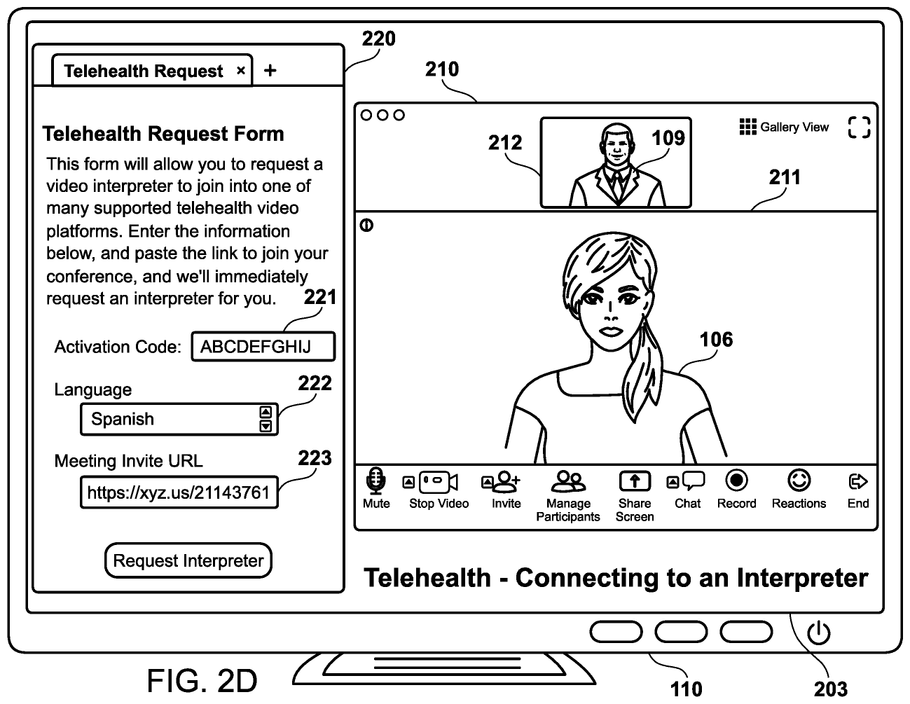

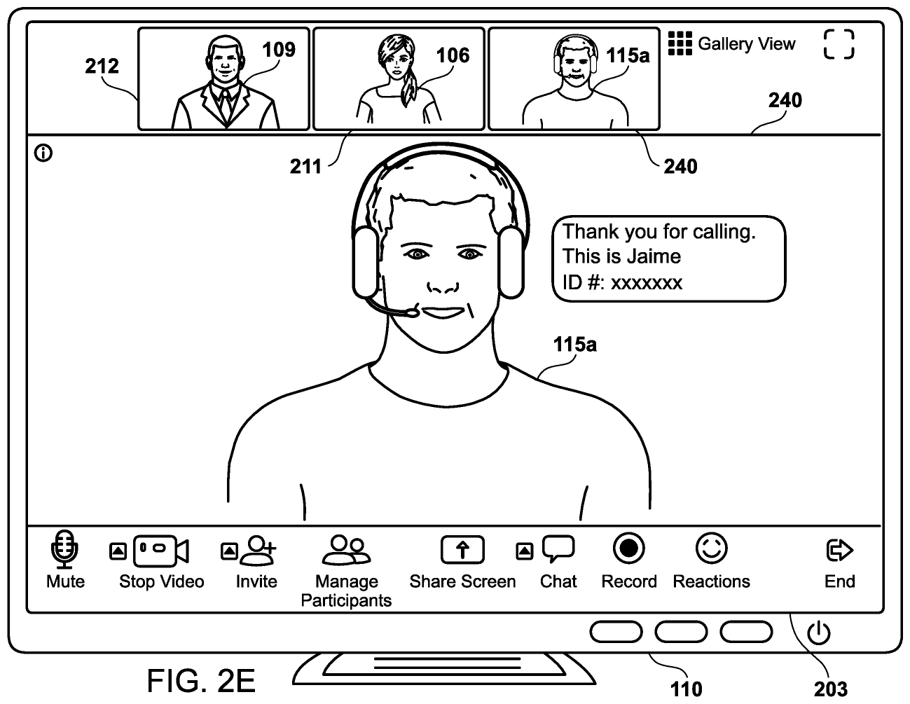

FIGS. 2A-2E illustrate examples of a sequence of screenshots rendered by the healthcare provider computing device 110 during the establishment and performance of the telehealth meeting. In particular, FIG. 2A illustrates an example of a telehealth GUI 201 being overlain over a messaging software application 202, such as an email client, on a display screen 203 of the healthcare provider computing device 110. The telehealth GUI 201 may be generated by a cloud-based software application that is hosted by the telehealth platform 101a, of which the healthcare provider computing device 110 is configured to operate. In essence, the health care provider 109 may schedule a teleconference via the messaging software application 202 with the patient 106 (e.g., sending an email), and then join the teleconference via the distinct, telehealth GUI 201. In an alternative embodiment, the messaging software application 202 may be integrated with the telehealth GUI 201, thereby allowing the healthcare provider 109 to both schedule a teleconference and participate in the teleconference from the same GUI.

Various menu indicia may be rendered by the telehealth GUI. For instance, a new meeting indicium 204, when activated, allows the healthcare provider 109 to establish a new telehealth conference. A schedule indicium 206 allows the healthcare provider 109 to schedule a teleconference. Upon the scheduled time for the teleconference, a join indicium 205 allows the healthcare provider 109 to join that telehealth conference. Optionally, a share screen indicium 207 allows the healthcare provider 109 to share his or her screen with other participants in the teleconference.

FIG. 2B illustrates an example of the messaging software application 202 allowing the healthcare provider 109 to send a message, such as an email to the patient 106 to schedule a teleconference. In essence, the messaging software application 202 allows the healthcare provider 109 to send a patient invite to the patient 106. (Email is just one example; other forms of communication (e.g., text message) may be utilized instead.) The patient invite may have various details specific to the scheduled, or potentially scheduled, teleconference, such as day and time of the teleconference, as well as a hyperlink that may be activated by the patient 106 to join the teleconference. The hyperlink may be specific to the particular telehealth platform 101a that is configured for use with the telehealth provider computing device 110.

Furthermore, FIG. 2C illustrates an example of a telehealth meeting GUI 210. In particular, the telehealth meeting GUI 210 may display various windows, each corresponding to a particular participant. For instance, the telehealth meeting GUI 210 may display a patient window 211 that displays a live video capture of the patient 106, and a healthcare provider window 212 that displays a live video capture of the healthcare provider 110. As an example, the healthcare provider 109 may expand the patient window 211 to better view the patient 106 during the teleconference; and vice versa.

During the teleconference, or based on previously known data such as from medical records of the patient 106, the healthcare provider 109 may learn that the patient 106 does not speak the same language. For example, the healthcare provider 109 may be an English-speaking physician that attempts to communicate with the patient 106, who may be a Spanish-speaking LEP. Accordingly, at the outset of the teleconference, or soon thereafter, the healthcare provider 109 may determine that a language interpreter is necessary to facilitate effective communication between himself or herself and the patient 106. Accordingly, FIG. 2D illustrates a language interpreter invite widget 220 that may be rendered in addition to the teleconference telehealth meeting GUI 210. As opposed to the telehealth meeting GUI 210, which is in active communication with the telehealth platform 101a to operate the telehealth meeting, the language interpreter invite widget 220 may be in communication with the language interpretation platform 130. As such, the language interpreter invite widget 220 is distinct from the telehealth meeting GUI 210. (Alternatively, the language interpreter invite widget 220 may be integrated within the telehealth meeting GUI 210, such as via a plugin.) The language interpreter invite widget 220 may have various fields, such as a customer account identifier field 221, a language request field 222, and a telehealth meeting identifier field 223. The customer account identifier field 221 may allow for various types of identifiers, such as an account number, an activation code corresponding to the account number, and the like. Furthermore, the language request field 222 may request an input for a language other than the native language of the geographical region in which the healthcare provider 109 is located. For example, if the healthcare provider 109 is located within the United States, the language request field may indicate languages other than English, such as Spanish, via a drop-down menu. (Alternatively, the requested language may be inputted.) Optionally, the language request field 222 may indicate both of the languages of the healthcare provider 109 and the patient 106. Furthermore, the telehealth meeting identifier field 223 may include data specific to the particular telehealth platform 101a that is configured for use with the healthcare provider computing device 110. (More, less, or different fields than those illustrated may be utilized by the language interpreter invite widget 220.)

The language interpreter invite widget 220 is illustrated as a web portal having a web form for the sake of illustration, but may be implemented based on one or more other modalities. For example, the language interpreter invite widget 220 may be integrated within an email message, a hyperlink, a phone call via an IVR, a mobile software application, a desktop tool tray software application, a text message, a calendar invitation, and a QR code. In essence, the language interpreter invite widget 220 allows the healthcare provider 109 to select from one of a number of different modalities to send an invite to a language interpreter during a telehealth meeting.

Finally, FIG. 2E illustrates the telehealth meeting GUI 210 depicting an updated telehealth meeting. In particular, the telehealth meeting GUI 210 illustrates an interpreter window 240 that displays a video capture of the available interpreter 115a. The healthcare provider 109 and the patient 106 may then proceed with the teleconference with the assistance of the language interpreter 115a performing live, language interpretation.

The GUIs particular to the specific telehealth platform 101a are provided only as examples. For example, the GUIs for the other telehealth platforms 101b-n each may have different GUIs from the GUIs rendered by the specific telehealth platform 101b-n.

In addition to allowing the healthcare provider 109 and/or the patient 106 to provide an interpreter request from one of a plurality of different modalities, the multi-modality electronic invite routing system 100 may provide a notification to the healthcare provider 109 and/or the patient 106 indicating the status of the next available language interpreter 115a, via the same modality or possibly a different modality. For example, the healthcare provider 109 may send an interpreter invite via email, and then receive a notification via email indicating that the next available language interpreter 115a has been found. As another example, the healthcare provider 109 may send an interpreter invite via a web form, and then receive a notification via email indicating that the next available language interpreter 115a has been found.

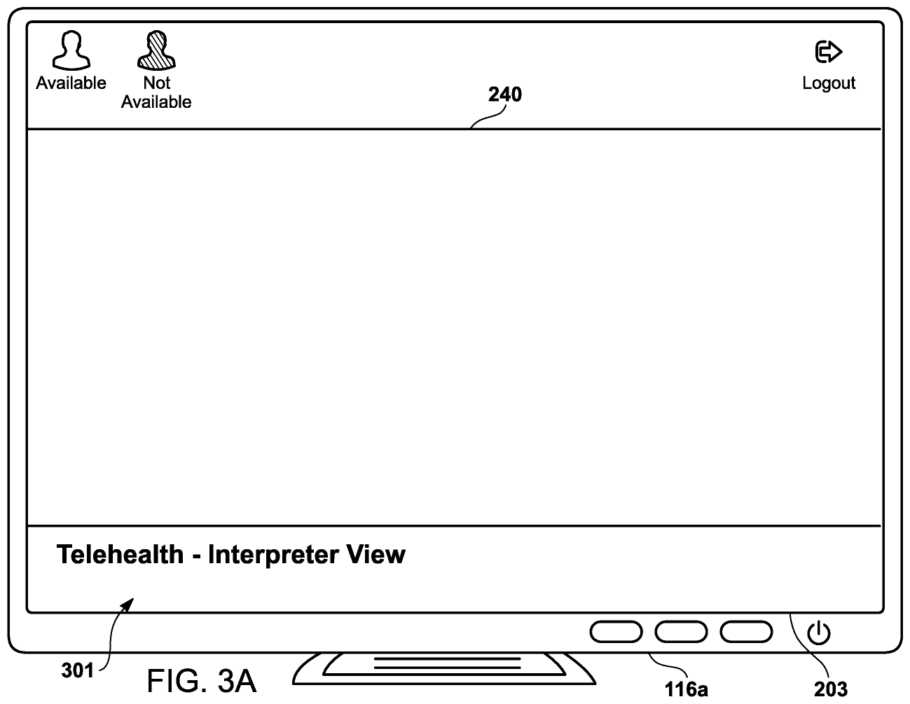

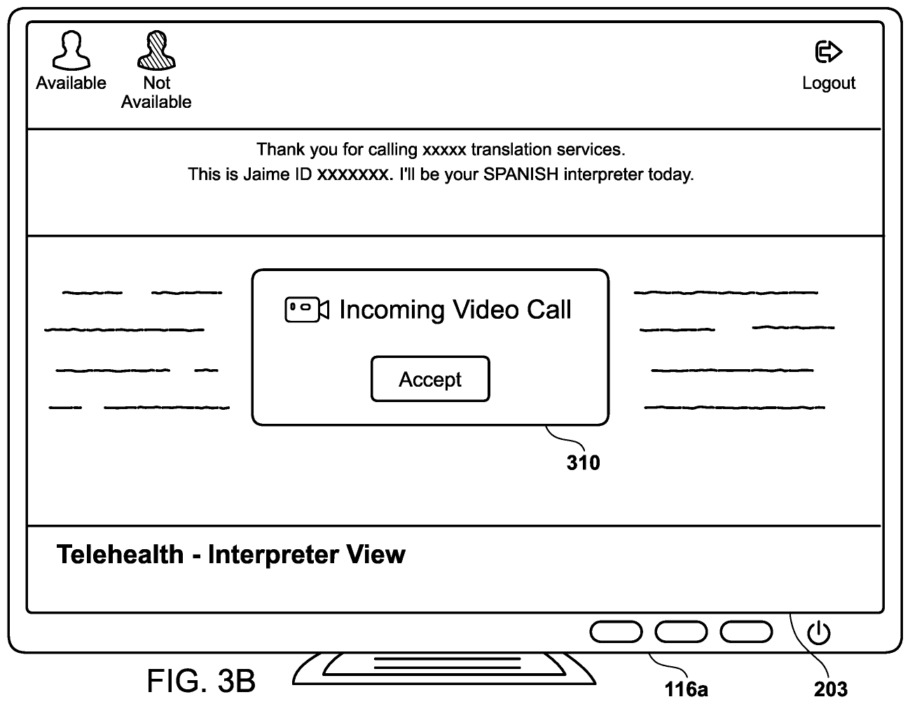

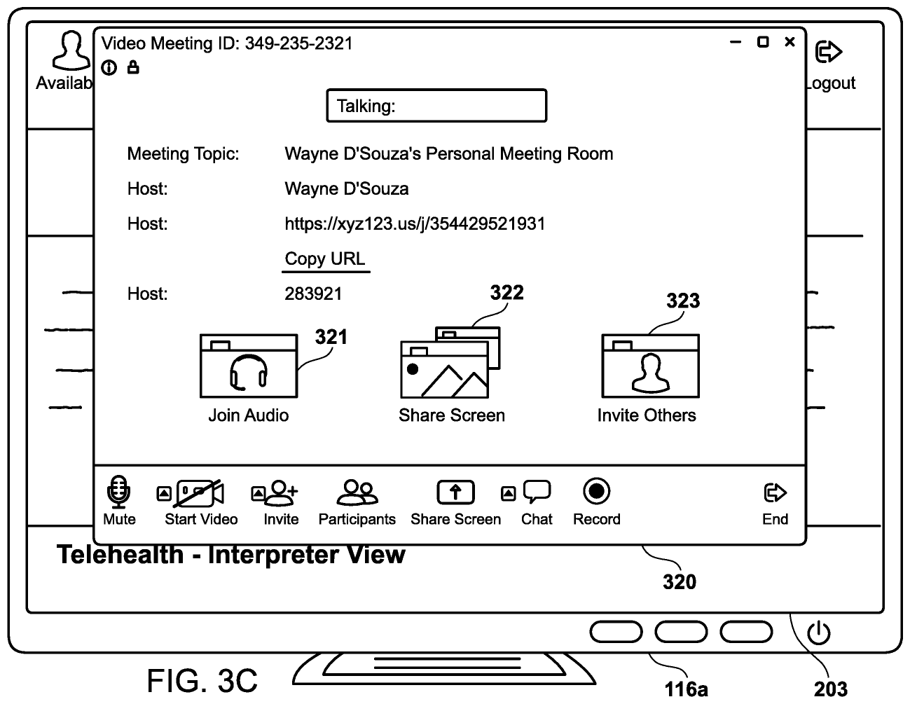

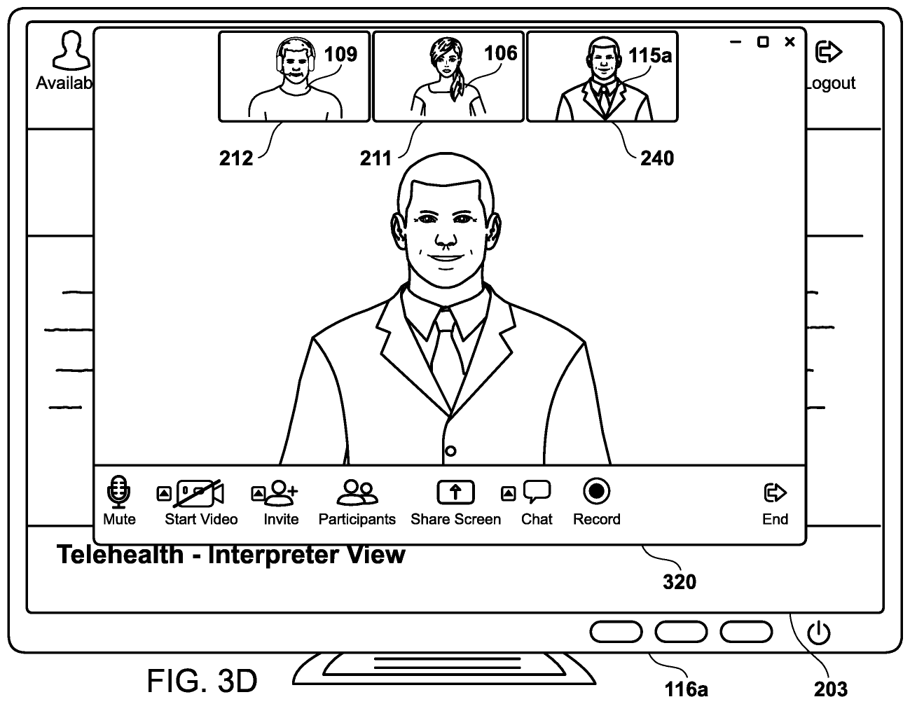

FIGS. 3A-3D illustrate examples of a sequence of screenshots rendered by the interpreter computing device 116a during the establishment and performance of the virtual telehealth meeting. In particular, FIG. 3A illustrates an interpreter GUI 301 that may be rendered on the interpreter computing device 116a to allow the language interpreter 115a to perform language interpretation via a telehealth virtual meeting. Via various indicia, the language interpreter 115a may indicate availability to participate in a telehealth conference. If available, the language interpreter 115a may receive an interpreter invite, as indicated by the interpreter invite notification 310 in the GUI 301 illustrated in FIG. 3B. Furthermore, as illustrated by FIG. 3C, an interpreter teleconference window 320 is automatically rendered upon acceptance of the interpreter invite (e.g., clicking an accept indicium 311) illustrated in the interpreter invite notification 310 of FIG. 3B. In other words, irrespective of one of the many different modalities through which the interpreter invite could be sent by the healthcare provider 109, the language interpreter 115a may view the interpreter invite notification 310 and the interpreter teleconference window 320 according to the same normalized format (e.g., a normalized GUI). In the interpreter teleconference window 320, the language interpreter 115a may select from various indicia to perform actions regarding the teleconference. For example, the language interpreter 115a may activate a join audio indicium 321, a share screen indicium 322, and an invite others indicium 323. Finally, FIG. 3D illustrates the interpreter window 240, the healthcare provider window 212, and the patient window 211 that are rendered during the updated telehealth conference after the joining of the language interpreter 115a.

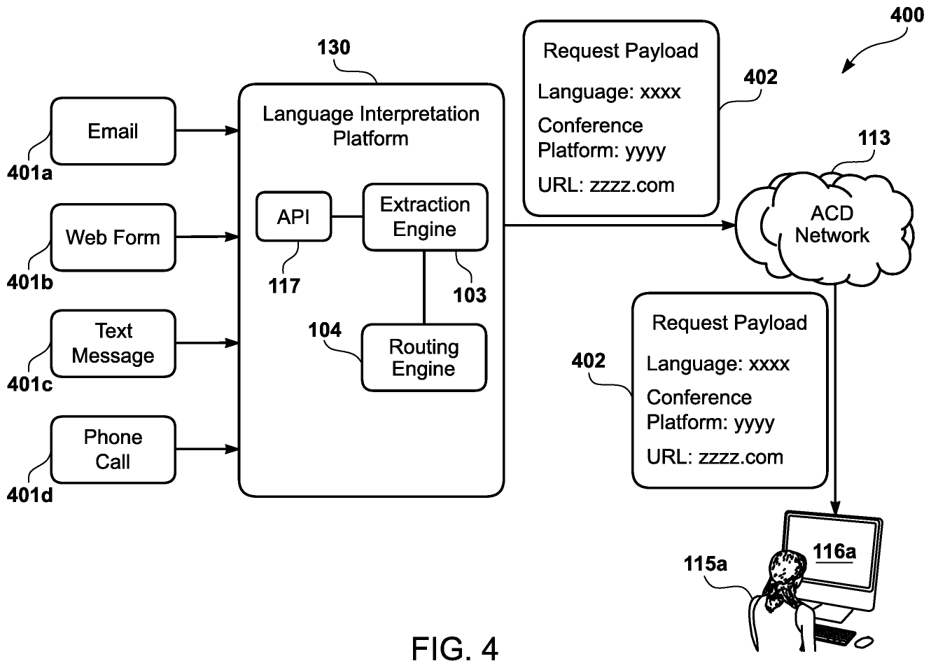

FIG. 4 illustrates an extraction configuration 400 in which the language interpretation platform 130 performs extraction of an interpreter request payload 402 from the interpreter invite that is sent from the healthcare provider computing system 110. Given that data for the interpreter invite may be presented in different formats from different modalities, the healthcare provider computing device 110 may make one or more function calls to the API 117 to invoke an extraction engine 103 to extract the pertinent data for normalization across the various modalities, thereby allowing for the universal telehealth interpreter interface illustrated in FIGS. 3A-3D. As examples, the language interpretation platform 130 may receive the interpreter invite via a plurality of modalities, such as an email modality 401a, a web form modality 401b, a text message modality 401c, and a phone call modality 401d (e.g., an invite fronted by an IVR). (The foregoing examples are provided only for illustrative purposes, given that additional or different modalities may be used instead.) For instance, in the case of the email modality 401a, the extraction engine 103 may parse an email to determine necessary information (e.g., customer account identifier, language request, telehealth meeting identifier, etc.) to join a telehealth conference. As an example, the extraction engine 103 may parse an email based on the sender (from) and recipient (to) addresses to determine the account and language, and then parse the body of the email for the telehealth meeting identifier. As an example, an email address such as “language.account@domain.com” may be parsed to determine the customer and/or language. The combination of the sender address and the recipient address may be mapped to determine which customer account the communication originated from. To avoid changes in email formats, a plurality of rules corresponding to the extraction engine may be encapsulated in an adapter to translate between the code of different systems. Furthermore, the routing engine 113 routes the request payload through the ACD network 104 to the interpreter workstation 116a. Furthermore, the extraction engine 103 may receive concurrent requests for interpreter invites of different modalities, and is able to simultaneously, or substantially simultaneously (i.e., without a perceivable human delay from the time of receipt to the time of processing), extract the interpreter payload request from each of the different modality requests.

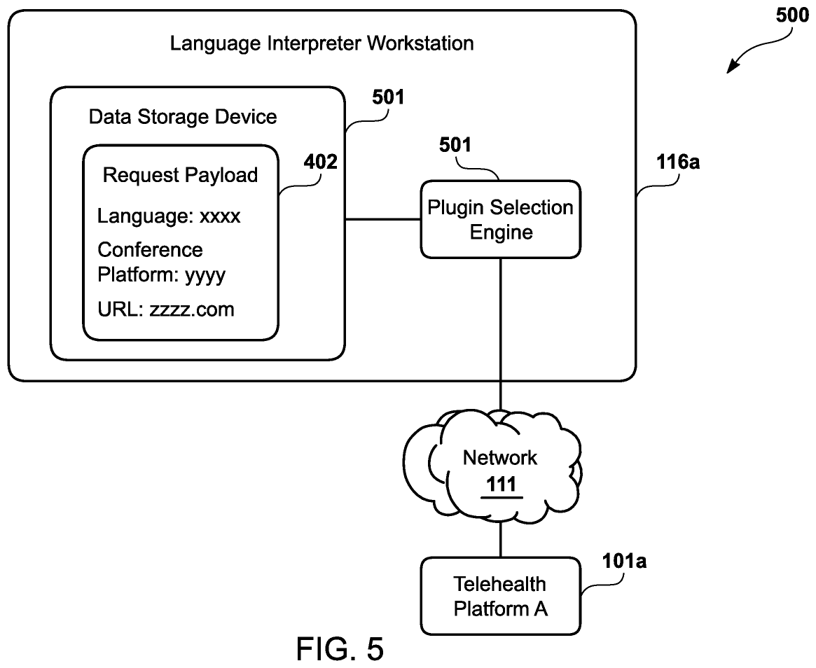

Additionally, FIG. 5 illustrates a plugin selection configuration 500 that allows the interpreter workstation 116a to automatically select, utilizing a plugin selection engine 501, a plugin corresponding to the telehealth platform 101a that is configured for use with the healthcare provider computing system 110. In other words, on a daily basis, the interpreter workstation 116a may receive many requests from different healthcare provider systems, and the plugin selection configuration 500 conveniently selects and invokes the necessary plugin, thereby avoiding the language interpreter having to switch between different telehealth platforms. Instead, the normalized request payload 402 allows the language interpreter computing device 116a to automatically determine the corresponding plugin associated with the telehealth platform 101a and join the telehealth conference.

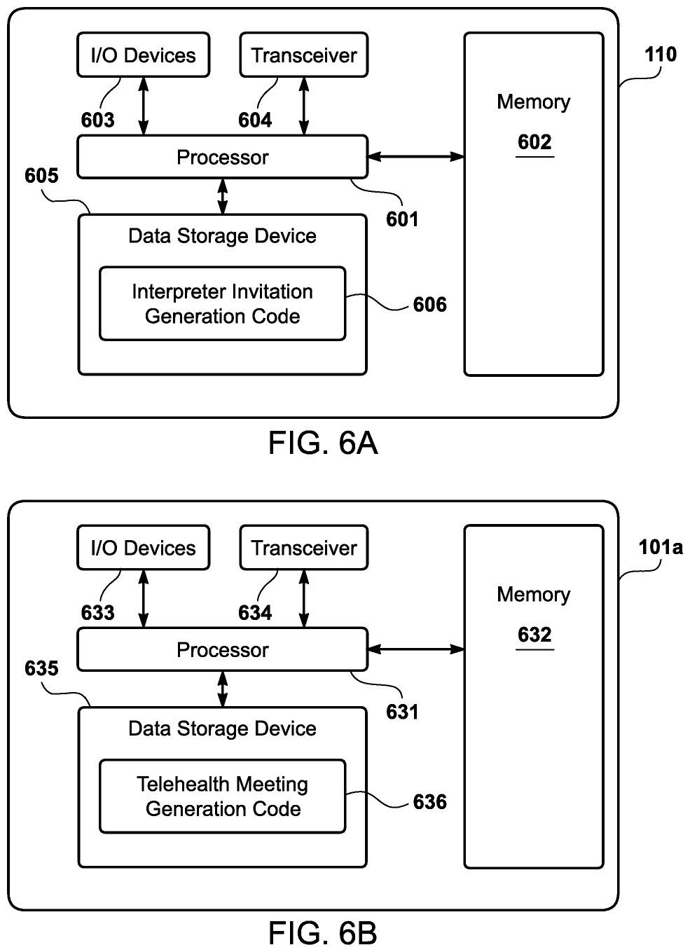

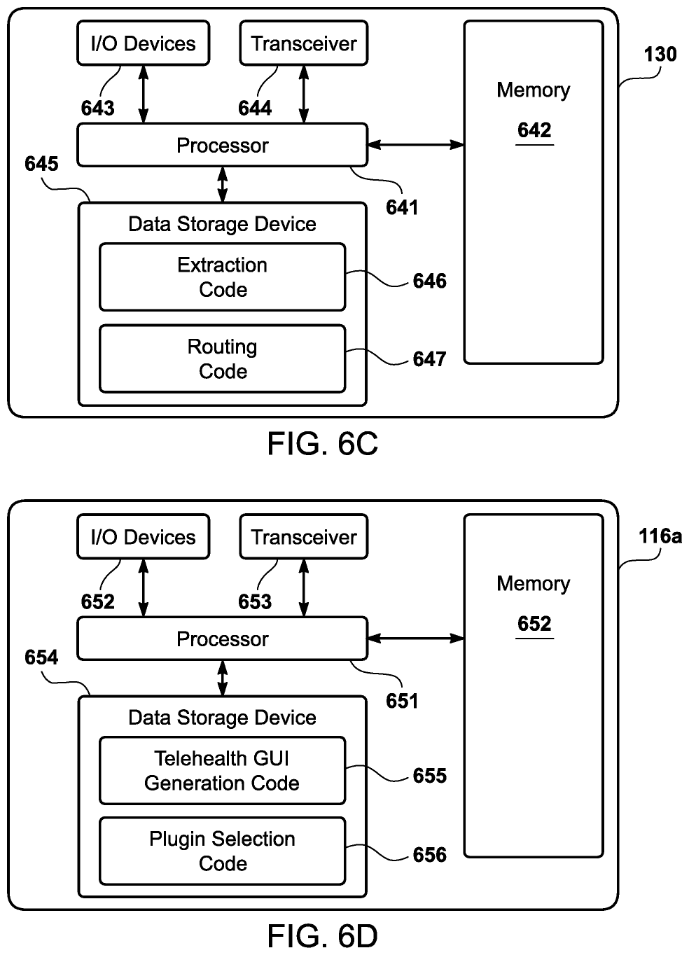

FIGS. 6A-6D illustrate various system configurations. In particular, FIG. 6A illustrates a system configuration for the healthcare provider computing device 110. A processor 601 may be specialized for invite generation. The system configuration may also include a memory device 602, which may temporarily store computer readable instructions performed by the processor 601. As an example of such computer readable instructions, a data storage device 606 within the system configuration may store interpreter invitation code 606, such as the language interpretation widget 220. Various devices (e.g., keyboard, microphone, mouse, pointing device, hand controller, joystick, display screen, holographic projector, etc.) may be used for input/output (“I/O”) devices 603. The system configuration may also have a transceiver 604 to send and receive data. Alternatively, a separate transmitter and receiver may be used instead.

Furthermore, FIG. 6B illustrates a system configuration for the telehealth platform 101a. A processor 631 may be specialized for performing telehealth meetings. The system configuration may also include a memory device 632, which may temporarily store computer readable instructions performed by the processor 631. As an example of such computer readable instructions, a data storage device 635 within the system configuration may store telehealth meeting generation code 636. Various devices (e.g., keyboard, microphone, mouse, pointing device, hand controller, joystick, display screen, holographic projector, etc.) may be used for I/O devices 633. The system configuration may also have a transceiver 634 to send and receive data. Alternatively, a separate transmitter and receiver may be used instead.

Furthermore, FIG. 6C illustrates a system configuration for the language interpretation platform 130. A processor 641 may be specialized for performing extraction of the request payload and routing to a language interpreter. The system configuration may also include a memory device 642, which may temporarily store computer readable instructions performed by the processor 641. As an example of such computer readable instructions, a data storage device 645 within the system configuration may store extraction code 646 to invoke the extraction engine 103 and routing code 647 to invoke the routing engine 104. Various devices (e.g., keyboard, microphone, mouse, pointing device, hand controller, joystick, display screen, holographic projector, etc.) may be used for I/O devices 643. The system configuration may also have a transceiver 644 to send and receive data. Alternatively, a separate transmitter and receiver may be used instead.

Additionally, FIG. 6D illustrates a system configuration for the language interpreter workstation 116a. A processor 651 may be specialized for generating a language interpreter interface and joining a language interpreter to a telehealth meeting. The system configuration may also include a memory device 652, which may temporarily store computer readable instructions performed by the processor 651. As an example of such computer readable instructions, a data storage device 654 within the system configuration may store telehealth GUI generation code 655 to generate a telehealth GUI and plugin selection code 656 to invoke the plugin selection engine 501. Various devices (e.g., keyboard, microphone, mouse, pointing device, hand controller, joystick, display screen, holographic projector, etc.) may be used for I/O devices 652. The system configuration may also have a transceiver 653 to send and receive data. Alternatively, a separate transmitter and receiver may be used instead.

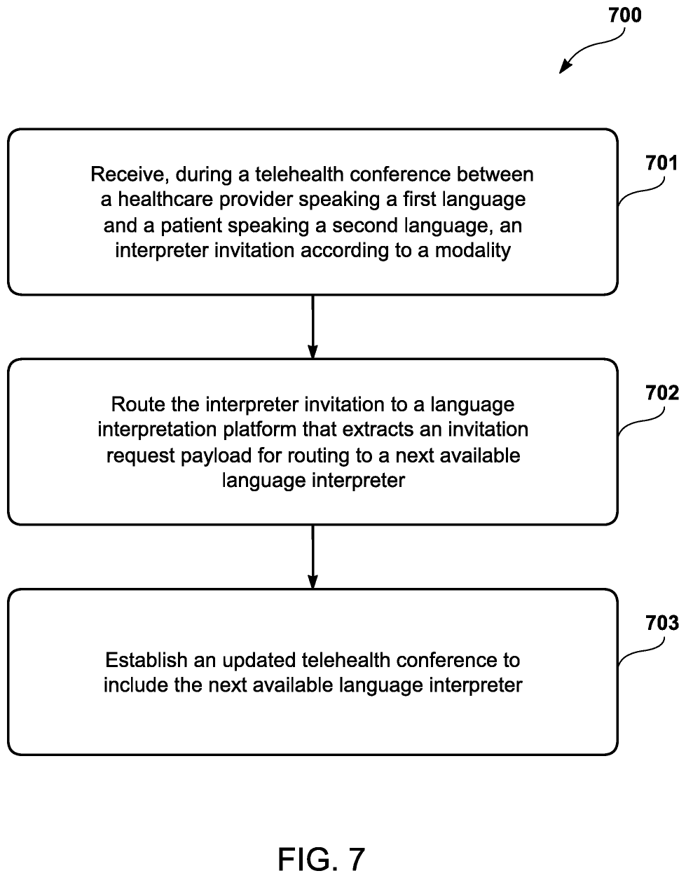

FIG. 7 illustrates a process 700 that may be utilized to provide multi-modality electronic invite routing. At a process block 701, the process 700 receives, during a telehealth conference between a healthcare provider 109 speaking a first language and a patient 106 speaking a second language, an interpreter invitation according to a modality generated through a user interface at a provider computing device operated by the healthcare provider. Furthermore, at a process block 702, the process 700 routes the interpreter invitation from the telehealth platform to a language interpretation platform that extracts an invitation request payload from the interpreter invitation and routes the invitation request payload, through an ACD network, to a next available language interpreter operating a language interpreter workstation such that the language interpreter workstation automatically selects a plugin according to a teleconference platform indicated by the invitation request payload. Finally, at a process block 703, the process 700 establishes an updated telehealth conference that modifies the telehealth conference to include the next available language interpreter to perform real-time language interpretation for a communication between the healthcare provider and the patient.

It is understood that the apparatuses, systems, computer program products, and processes described herein may also be applied in other types of apparatuses, systems, computer program products, and processes. Those skilled in the art will appreciate that the various adaptations and modifications of the embodiments of the apparatuses, systems, computer program products, and processes described herein may be configured without departing from the scope and spirit of the present apparatuses, systems, computer program products, and processes. Therefore, it is to be understood that, within the scope of the appended claims, the present apparatuses, systems, computer program products, and processes may be practiced other than as specifically described herein.

We claim:

1. A computer-implemented process comprising:
receiving, during a telehealth conference between a healthcare provider speaking a first language and a patient speaking a second language, an interpreter invitation according to a modality generated through a user interface at a provider computing device operated by the healthcare provider;
routing the interpreter invitation from the telehealth platform to a language interpretation platform that extracts an invitation request payload from the interpreter invitation and routes the invitation request payload, through an automatic call distribution network, to a next available language interpreter operating a language interpreter workstation such that the language interpreter workstation automatically selects a plugin according to a teleconference platform indicated by the invitation request payload; and
establishing an updated telehealth conference that modifies the telehealth conference to include the next available language interpreter to perform real-time language interpretation for a communication between the healthcare provider and the patient.

2. The computer-implemented process of claim 1, wherein the modality is selected from the group consisting of: a web form, an email, a hyperlink, a phone call via an interactive voice recognition system, a mobile software application, a desktop tool tray software application, a text message, a calendar invitation, a QR code.

3. The computer-implemented process of claim 1, wherein the invitation request payload comprises a customer identifier, a language request, and a telehealth meeting identifier.

4. The computer-implemented process of claim 1, wherein the language interpreter workstation automatically performs the selection of the plugin by automatically deciphering the invitation request payload, automatically recognizing the telehealth platform based on the automatic deciphering, and automatically launching a corresponding software application that automatically joints the next available language interpreter to the updated telehealth conference.

5. The computer-implemented process of claim 1, wherein the language interpretation platform monitors a start and end time of the updated telehealth conference to determine billing for the real-time language interpretation.

6. The computer-implemented process of claim 1, wherein the language interpretation platform monitors a start and end time of the updated telehealth conference to update availability of the next available language interpreter.

7. The computer-implemented process of claim 1, wherein the language interpretation platform monitors the updated telehealth conference, via an audio-only system, to maintain quality control of the real-time language interpretation.

8. The computer-implemented process of claim 1, wherein the updated telehealth conference joins the next available language interpreter in at least substantially real-time from a time when the interpreter invitation is received.

9. The computer-implemented process of claim 1, wherein the updated telehealth conference joins the next available language interpreter based on a reservation.

10. The computer-implemented process of claim 1, wherein the first language and the second language are human-spoken languages.

11. The computer-implemented process of claim 1, wherein one of the first language and the second language is sign language.

12. The computer-implemented process of claim 1, wherein the teleconference platform is a video-based platform.

13. The computer-implemented process of claim 1, wherein the next available language interpreter is a human language interpreter.

14. The computer-implemented process of claim 1, wherein the next available language interpreter is a machine language interpreter.

15. A computer-implemented system comprising:
a receiver that receives, during a telehealth conference between a healthcare provider speaking a first human-spoken language and a patient speaking a second human-spoken language, an interpreter invitation according to a modality generated through a user interface at a provider computing device operated by the healthcare provider;
a routing engine that routes the interpreter invitation from the telehealth platform to a language interpretation platform that extracts an invitation request payload from the interpreter invitation and routes the invitation request payload, through an automatic call distribution network, to a next available language interpreter operating a language interpreter workstation such that the language interpreter workstation automatically selects a plugin according to a video-based teleconference platform indicated by the invitation request payload; and
a processor that establishes an updated video-based telehealth conference that modifies the video-based telehealth conference to include the next available language interpreter to perform real-time language interpretation for a communication between the healthcare provider and the patient.

16. The computer-implemented system of claim 15, wherein the modality is selected from the group consisting of: a web form, an email, a hyperlink, a phone call via an interactive voice recognition system, a mobile software application, a desktop tool tray software application, a text message, a calendar invitation, a QR code.

17. The computer-implemented system of claim 15, wherein the invitation request payload comprises a customer identifier, a language request, and a telehealth meeting identifier.

18. The computer-implemented system of claim 15, wherein the updated video-based telehealth conference joins the next available language interpreter in at least substantially real-time from a time when the interpreter invitation is received.

19. The computer-implemented process of claim 15, wherein the updated video-based telehealth conference joins the next available language interpreter based on a reservation.

20. The computer-implemented process of claim 15, wherein the next available language interpreter is a human language interpreter.
